(19) United States
(12) Patent Application Publication
(10) Pub. No.: US 2003/0125785 A1
(43) Pub. Date: Jul. 3, 2003

(54) Disconnection system for two-part cochlear electrode and method of making same

(76) Inventors: Janusz A. Kuzma, Parker, CO (US); Thomas J. Balkany, Coral Gables, FL (US)

Correspondence Address: ADVANCED BIONICS CORPORATION, 12740 SAN FERNANDO ROAD, SYLMAR, CA, 91342, US

(21) Appl. No.: 10/116,449

(22) Filed: Apr. 3, 2002

Related U.S. Application Data
(60) Provisional application No. 60/282,646, filed on Apr. 9, 2001.

(51) Int. Cl. A61N 1/05

(52) U.S. Cl. 607/116

(57) ABSTRACT

A disconnection system (10) for use with a cochlear electrode (20) and positioner (30) includes a cuff or band (12) that is placed and secured, e.g., glued, to the electrode carrier near its distal tip (21). A distal tip (31) of the positioner (30), or in one embodiment a flat strip (52) protruding from the distal tip, is readily slid into the cuff, so as to be engaged thereby. Forward longitudinal (pushing) forces exerted on the positioner during the insertion process maintain the distal tip of the positioner securely engaged by the cuff. Should it subsequently become necessary to remove the positioner (30), then a gentle rearward longitudinal (pulling) force may be readily applied to the positioner, causing the distal tip (31) of the positioner (30) to pull away from the cuff (12), thereby disconnecting the positioner (30) from the electrode (20).

The present application claims the benefit of U.S. Provisional Application Serial No. 60/282,646, filed Apr. 9, 2001, which application is incorporated herein by reference.

### BACKGROUND OF THE INVENTION

The present invention relates to implantable lead systems used with, e.g., an implantable cochlear stimulator. More particularly, the invention relates to a disconnection system for a two-part electrode system, and a method of making such a system.

Cochlear electrodes are generally built in the form of elongated, flexible polymer structures with array contacts and connecting wires. It is generally understood that the best performance from a cochlear implant is achieved with the electrode contacts on the cochlear electrode are facing and closely positioned to the modiolar wall of the cochlea.

There are two main types of electrode systems designed to place the electrode contacts in perimodiolar (near or around the modiolus) position. The first is as described in U.S. Pat. No. 6,038,484 or 6,195,586, both of which patents are incorporated herein by reference. Such electrode system incorporates an electrode carrier and a positioner, which positioner helps hold and/or position the electrodes against the modiolar wall. The second is as described in U.S. Pat. No. 6,125,302, also incorporated herein by reference, which electrode system employs a pre-curved electrode carrier that is straightened through the use of a stylet wire during the insertion process, but which assumes its precurved shape once the stylet is removed.

To reduce damage to the cochlea structure, electrode carriers are made from very soft, flexible material, and because of this characteristic, they are generally difficult to insert into the cochlea without the use of special supporting tools. An example of such insertion tools is described in U.S. Pat. Nos. 5,443,493 and 6,149,657, both of which patents are also incorporated herein by reference. Another example of an insertion tool is as described in applicants' co-pending U.S. patent application Ser. No. ______, filed concurrently herewith (Attorney Docket No. AB-235U), entitled “Insertion Tool For Placement of Electrode/Positioner System Inside the Cochlear Lumen”, which application is also incorporated herein by reference.

The present invention is particularly applicable to the first type of electrode system mentioned above, i.e., one wherein a positioner is used in conjunction with the electrode carrier in order to help position the electrode carrier so that the electrode contacts reside against, or near, the modiolar wall. In such a system, it is helpful during the insertion process if a distal tip of the positioner is attached to the electrode carrier near (but not at) the distal tip of the carrier, and if the electrode carrier and positioner are then inserted into the cochlea at the same time, with the positioner joined to the carrier as described. Such an electrode/positioner system is used by Advanced Bionics Corporation, of Sylmar, Calif., and is known as the “HiFocus II” electrode. The HiFocus II electrode is as described in U.S. Pat. Nos. 6,195,586 and/or 6,321,125, both of which patents are incorporated herein by reference.

In some instances, although it is desirable to have the distal tip of the positioner attached to the cochlear electrode carrier during the insertion process, it is also desirable to have the positioner detached from the electrode carrier in the event the positioner needs to be subsequently removed. Such detachment can be particularly important in the event that the cochlea has become ossified and only the electrode array can be used, or in some instances of explantation. An example of one type of cochlear electrode system that includes a detachable flexible positioner is described in applicant Kuzma's copending U.S. patent application Ser. No. 09/443/627, filed Nov. 19, 1999, which application is incorporated herein by reference.

The present invention relates to an improved electrode system wherein the positioner is attached to the electrode carrier as a standard configuration, but wherein the positioner can be easily detached from the electrode array when needed, e.g., to allow insertion of the electrode array without a positioner (for example, into a narrow ossified cochlea), or during explantation if scare tissue has formed between the positioner and the electrode array.

### SUMMARY OF THE INVENTION

The present invention provides an electrode/positioner system having a disconnection system that allows the positioner to be readily and gently disconnected from the electrode and withdrawn from the cochlea, if necessary. Such disconnection may be required, e.g., to allow insertion of the electrode array without a positioner (for example, into a narrow ossified cochlea), or during explantation in the event that scare tissue has formed between the positioner and the electrode array.

In accordance with one aspect of the invention, the present invention allows a cochlear electrode to be manufactured as a separate element, apart from the manufacture of the positioner, and vice-versa, thereby simplifying the manufacturing process and reducing costs. That is, an electrode, e.g., a HiFocus electrode of the type described in U.S. Pat. No. 6,129,753, may be readily and inexpensively manufactured; and a positioner adapted for use with such HiFocus electrode may also be readily and inexpensively made as taught, e.g., in U.S. Pat. No. 6,078,841. Both the '753 and '841 patents are incorporated herein by reference. Then, the disconnection system of the present invention may be used to allow the positioner to be detachably secured to the electrode during the manufacturing and/or insertion process, and to allow a subsequent gentle disconnection of the positioner from the electrode, if necessary.

The disconnection system of the present invention comprises, in one embodiment, a stretchable cuff that is placed and secured, e.g., glued, around the electrode carrier near its distal tip, e.g., at a location between the third and fourth electrode contacts, using a positioning tool as described herein. Once the cuff is thus positioned near the distal tip of the electrode carrier, a distal tip of the positioner, or a flat strip protruding from the distal tip, may be readily slid into the cuff, so as to be engaged thereby. A forward longitudinal force (i.e., a pushing force) exerted on the positioner during the insertion process causes the positioner to remain securely engaged by the cuff throughout the insertion process. However, should it subsequently become necessary to remove the positioner, then a rearward longitudinal force (i.e., a pulling force) may be gently applied to cause the distal tip of the positioner to pull away from the cuff, thereby freeing or disconnecting the positioner from the electrode.

Advantageously, the disconnection system of the present invention allows a gentle disconnection to occur between the electrode and positioner during the removal process. Such a gentle disconnection is highly advantageous because it allows the positioner and/or the electrode array to be removed from the cochlea should re-implantation be necessary without seriously damaging the scar tissue that may have formed in the cochlea in the space between the positioner and electrode. Should re-implantation be necessary, the new electrode array may be easily slid into the space vacated by the removed electrode array, and a separate positioner, or no positioner, may be used with such new electrode array.

### BRIEF DESCRIPTION OF THE DRAWINGS

The above and other aspects, features and advantages of the present invention will be more apparent from the following more particular description thereof, presented in conjunction with the following drawings wherein:

FIG. 1 shows the cuff disconnection system of the present invention with a positioner held by the cuff to the electrode;

FIG. 2A illustrates the manner in which the distal tip of the positioner, in one embodiment, is inserted into the cuff prior to the insertion process through application of a forward longitudinal (pushing) force on the positioner;

FIG. 2B illustrates the manner in which the positioner is removed from the cuff through application of a rearward longitudinal (pulling) force;

FIG. 3 illustrates a tool used to place the cuff near the distal end of the electrode;

FIG. 4 shows an enlarged view of a distal tip of the tool of FIG. 3, depicting fingers of the tool holding a cuff in an expanded or open position suitable for placing the cuff at its desired position near the distal tip of the electrode;

FIG. 5 is a side sectional view that illustrates placing the cuff at its desired location near the distal end of the electrode using the tool of FIG. 3;

FIG. 6 is a perspective view of an alternative embodiment of a cuff disconnection system made in accordance with the invention;

FIGS. 7A, 7B and 7C are a side view of the alternate embodiment of the invention shown in FIG. 6, with FIG. 7B being an enlarged view of the connection mechanism shown in FIG. 7A and FIG. 6, and with FIG. 7C illustrating how the positioner is disconnected from a cuff carried on the electrode; and

FIGS. 8A and 8B illustrate a preferred manner of making the embodiment of the invention shown in FIG. 6.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing the general principles of the invention. The scope of the invention should be determined with reference to the claims.

The present invention relates to the detachable securement of the proximal end of a positioner near the distal end of an electrode. For purposes of the present invention, the terms “electrode” and “electrode carrier” are used synonymously. The details associated with the manufacture and use of the positioner and electrode (or electrode carrier) will not be presented herein, as they may be found in the referenced patents and patent applications.

Further, it is to be understood that although the description that follows shows an electrode/positioner system adapted for insertion into a human cochlea, which represents a preferred application of the invention, the invention may be used for other applications as well. That is, the invention may be used with any two-part electrode/positioner system adapted for insertion into a body cavity or tissue.

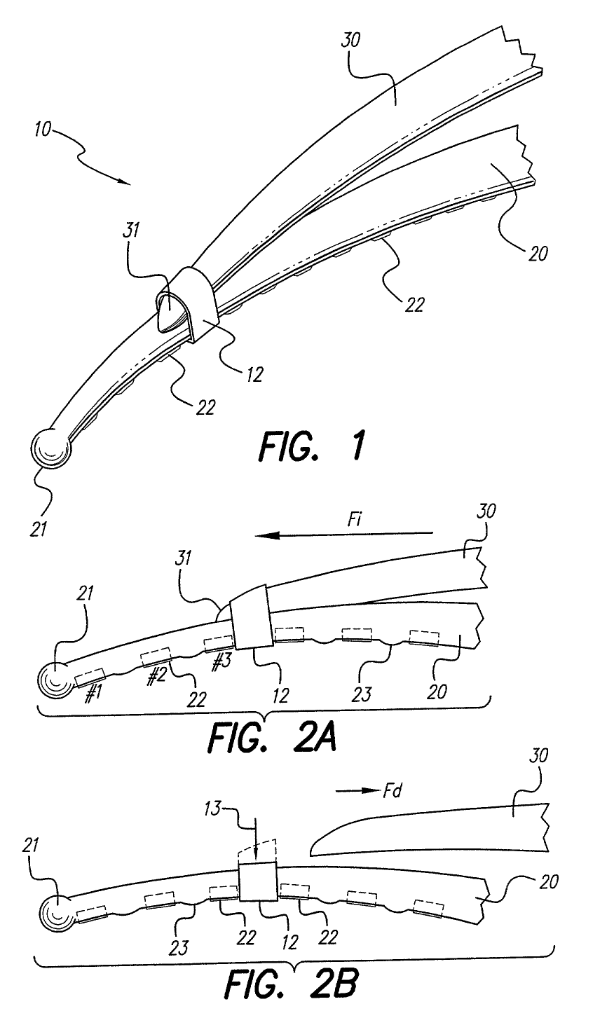

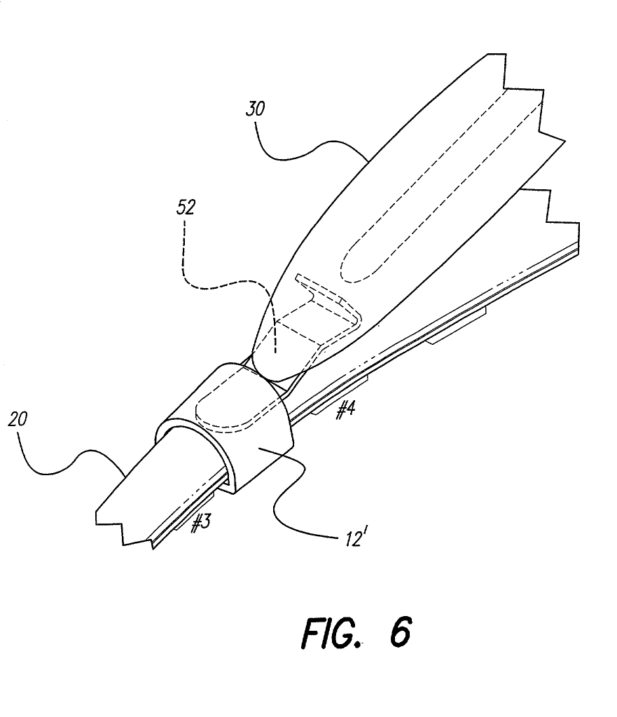

FIG. 1 shows a cuff disconnection system 10 made in accordance with the present invention. The system 10 includes a cuff 12 that is secured to an electrode 20 near, but not at, the distal tip 21 of the electrode 20. It should be noted that the cuff 12 may take many forms, such as a band or a loop or equivalent structure. The cuff 12 (or loop or band or other connecting structure) allows a distal tip of the positioner, or an element attached to the distal tip of the positioner, e.g., as shown and discussed below in conjunction with FIG. 6, to be engaged therewith when the positioner is moved in a forward direction, and to be disengaged therewith when the positioner is moved in a rearward direction.

The electrode 20 includes a multiplicity of spaced-apart electrode contacts 22 through which an electrical stimulus or stimuli may be applied to nearby tissue. In some embodiments, the electrode 20 may include small insulating bumps 23, or humps, between the respective electrode contacts 22. Typically, for the HiFocus electrode, there are sixteen electrode contacts 22, which electrode contacts 22 are spaced apart along one side of the electrode 20, however such number, positioning and location are only exemplary.

As indicated, the cuff 12 is preferably positioned on the electrode 20 at a location that is near, but not at, the distal tip 21 of the electrode 20. For the embodiment shown in FIG. 1, the cuff 12 is positioned at a location that is between the #3 and #4 electrode contacts 22, where the #1 electrode contact is the most distal electrode contact (closest to the distal tip 21). Typically, for a cochlear electrode 20, the cuff 12 will be positioned at a distance that is between about 3 mm and 6 mm from the distal tip 21 of the electrode 20.

As seen in the embodiment of the invention shown in FIGS. 1 and 2A, a distal tip 31 of a positioner 30 is held securely in the cuff 12. Moreover, the distal tip 31 of the positioner 30 has a cross-sectional area that is tapered, thereby facilitating insertion of the tip 31 into the cuff, but preventing the body of the positioner 30 from passing through the cuff 12. Hence, when a distal longitudinal force (i.e., a force pushing towards the distal end of the electrode) Fi is applied, the tip 31 of the positioner 30 is securely held within the cuff 12. It should be noted that this sample principle applies, and may be used, even when the distal tip fo the positioner has a cross-sectional area that is not tapered.

The cuff 12 is preferably made from a section of thin-walled silastic tubing, having a wall thickness of about 100 to 150 microns. As such, the cuff is somewhat stretchable, much like a rubber band, and exerts a radial force 13 (FIG. 2B) which tends to hold the positioner securely within the cuff when stretched. In one embodiment, the un-stretched inside diameter (ID) of the band, or thin-walled tube from which the cuff 12 is made, is the same as, or only slightly less than, the outside diameter (OD) of the electrode. Hence, the distal tip 31 of the positioner may be easily pulled out of the band or cuff 12 with a gentle pulling force.

Should it ever become necessary to remove the positioner 30 from the cuff 12, and thereby detach the positioner from the electrode 20, then all that is required is to apply a rearward longitudinal (pulling) force Fd (a force that pulls the positioner towards a proximal end of the positioner), as illustrated in FIG. 2B. The force Fd, which typically may be much less than the force Fi, e.g., Fi≧10Fd, and may thus be characterized as a gentle force, readily pulls the tip 31 of the positioner out of the cuff 12, thereby separating the positioner 30 from the electrode 20. Such gentle separation force advantageously allows separation of the positioner 30 from the electrode 20 without significant trauma to the patient or scar tissue (e.g., ossified cochlea) within the patient's cochlea, thereby allowing removal of electrode and positioner and, as required, reinsertion of a new electrode.

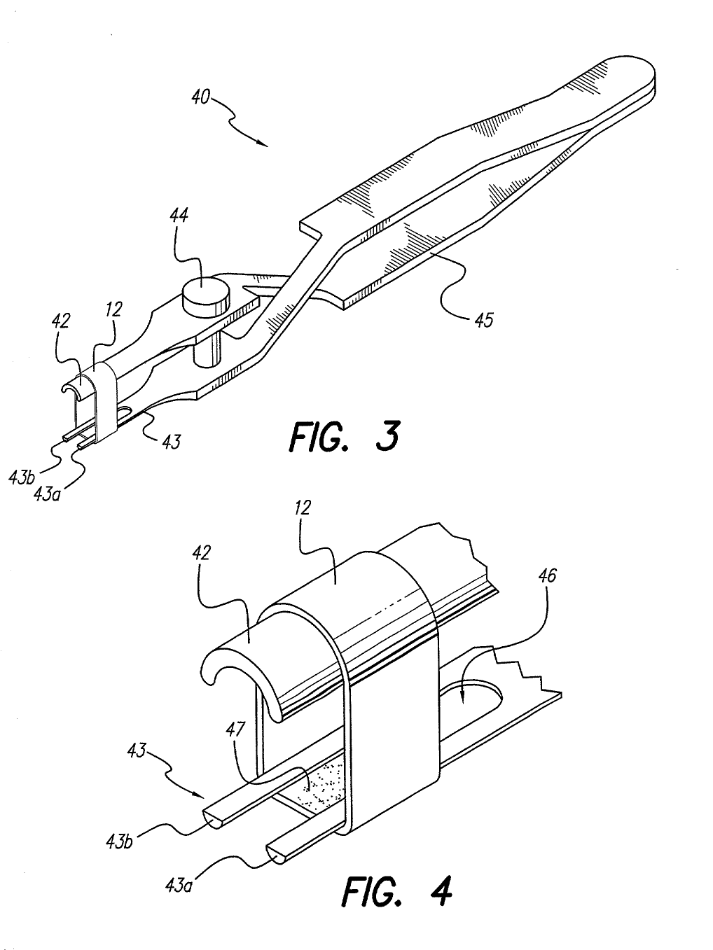

Turning next to FIG. 3, a tool 40 that may be used to place the cuff 12 near the distal end of the electrode 20 is illustrated. The tool 40 comprises reverse action tweezers, having a handle portion 45. When opposing sides of the handle portion 45 are pushed, or squeezed, together, fingers 42 and 43 at a distal end of the tool are forced to separate. A thumb screw 44 may be set so as to hold the separation of the fingers 42 and 43 at a desired separation distance when the squeezing force applied to the handle 45 is released.

As seen best in FIG. 4, the finger 43 is separated into two fingers 43a and 43b, thereby creating an opening or slot 46. When the cuff 12 is held by the expanded fingers 42 and 43, as shown in FIGS. 3 and 4, then a suitable bonding agent or glue 47, e.g., adhesive silicone, may be spread on an inside surface of the cuff 12 in the region of the slot 46.

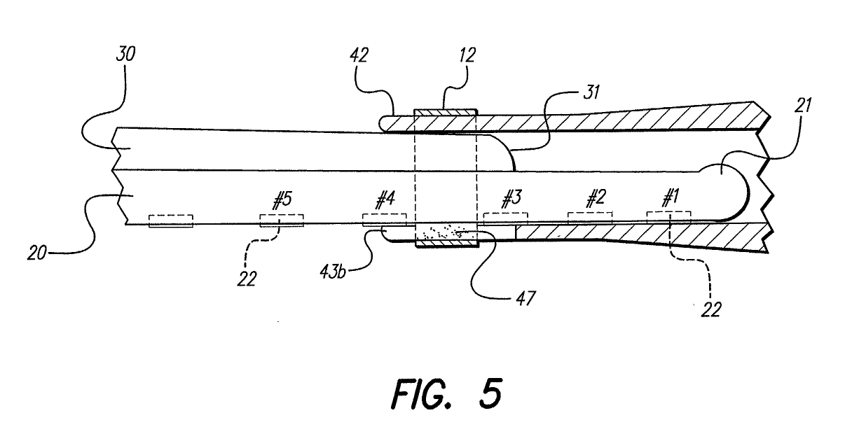

To place the cuff 12 at its desired location near the distal end of the electrode 20, the tool 40 is adjusted to hold the cuff 12 in an expanded position, as seen in FIG. 4 or FIG. 5. Then, while thus held, the distal tip 21 of the electrode is slid through the opening of the cuff 12 until the cuff is over the desired position near the distal tip 21 of the electrode. The distal tip 31 of a positioner 30 is likewise placed through the cuff 12. Then, the tool 40 is removed from the cuff, causing the fingers 42 and 43 to be withdrawn from the cuff, and the bonding glue 47 cures to hold the cuff 12 securely in its desired position relative to the electrode array.

It should be noted that FIG. 5 shows the cuff 12 fastened (glued) to the electrode 20 at a location that is between the #3 and #4 electrode contacts 22. Such fastening location is only exemplary.

An alternative embodiment of the invention is illustrated in FIGS. 6 through 8B. In this alternative embodiment, a cuff 12′ tightly encircles the electrode array 20 at a desired location, e.g., between the 3rd and 4th electrode contacts. The positioner 30 has a flat strip 52 that protrudes from its distal end. The protruding strip 52 is preferably made from a biocompatible metal, e.g., platinum.

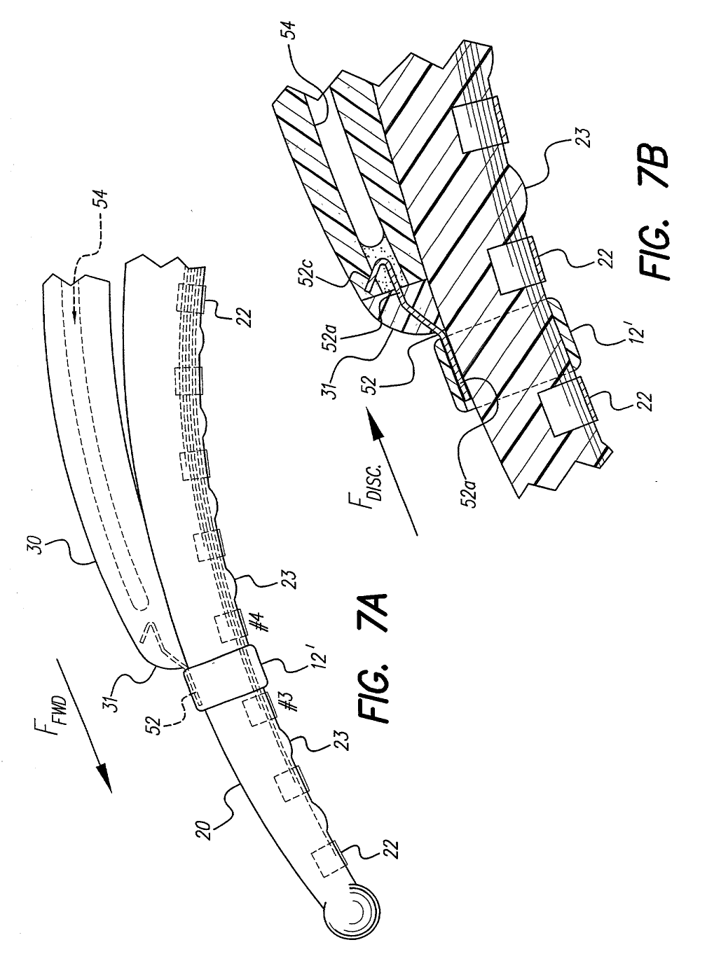

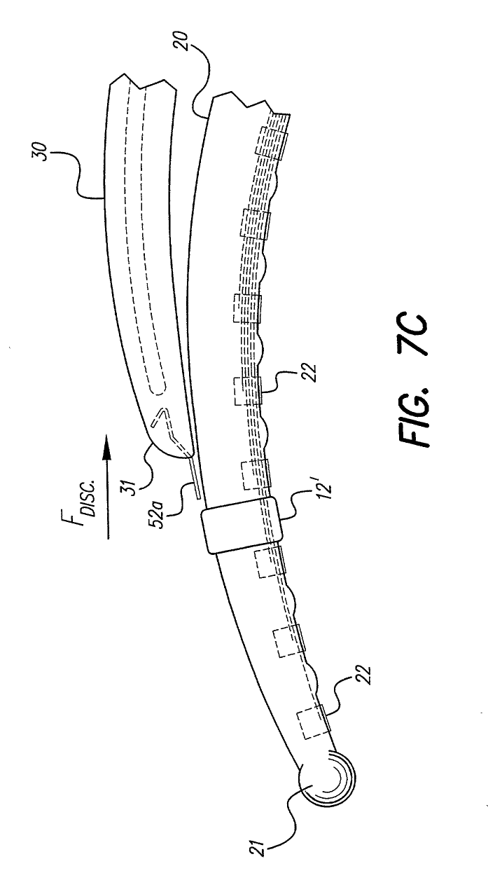

As seen best in FIGS. 7A, 7B and 7C, a distal end of the protruding flat strip 52 is adapted to slide under the cuff 12′. A proximal end of the stip 52 is embedded within the tip 31 of the positioner 30. The embedded portion of the strip 52 preferably includes a bend B that helps anchor the strip within the tip 31 of the positioner, thereby making it extremely difficult to ever allow the strip to be pulled out of the tip 31 of the positioner 30.

FIG. 7A illustrates the strip 52 engaged with the cuff 12′ as a forward force F_{FWD} is applied to the positioner. Such forward force is typically applied by first threading a lumen 54 that passes longitudinally through the body of the positioner on a stylet wire and then pushing the positioner off of the stylet wire into the cochlea, or other body cavity, with the aid of an insertion tool, such as the tool disclosed in applicants' co-pending patent application [Docket Ab-235U], Ser. No. ______, filed concurrently herewith, and incorporated herein by reference. During application of the force F_{FWD}, the strip 52 remains attached to, i.e., engaged with, the electrode 20 by means of the cuff 12′. Thus, the force F_{FWD} not only pushes the positioner 30 in the forward direction, but such force is also transferred to the electrode 20 and causes the electrode 20 to advance forward.

FIGS. 7B and 7C illustrate what happens when a disconnection or rearward force F_{DISC} is applied to the positioner. In FIG. 7B, the disconnection force F_{DISC} has just been applied, but the stip 52 has not yet been pulled out from the cuff 12′. In FIG. 7C, with the continued application of the force F_{DISC}, the strip 52 does pull out of the cuff 12′, and the positioner 30 is thus disconnected from the electrode 20.

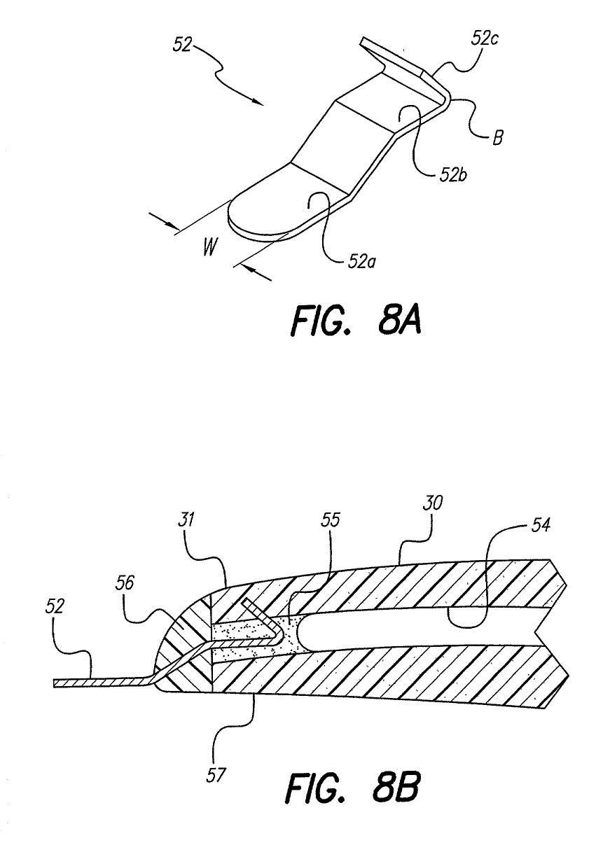

FIG. 8A illustrates a preferred flat strip 52 prior to embedding the stip into the distal tip 31 of a positioner. The strip 52 has a width W. For a cochlear postioner, the width W is about 0.30 mm. The strip has three main sections: a protruding portion 52a, an embedded portion 52b, and an anchoring portion 52c. The protruding portion 52a is substantially parallel with but offset from the embedded portion 52b. The protruding portion 52a is connected to the embedded body portion 52b by means of a slanting portion. The anchoring portion 52c is bent relative to the body portion 52b about 150-170 degrees.

FIG. 8B illustrates one manner in which the strip 52 may be embedded within the tip 31 of the positioner 30. The positioner 30 is made having a lumen 54 that passes longitudinally therethrough. A suitable amount of a liquid silicone material adhesive 55, such as LSR25, or equivalent, is placed in the distal end of the lumen 54. While the material 55 is still in a semi-liquid state, e.g., prior to its curing, the body strip portion 52b and anchoring portion 52c are inserted into the lumen 55 so as to be fully embedded within the not-yet cured material 55. Then, an additional amount of silicone or similar material 56 is placed over the end of the positioner 31 so as to completely surround the offset portion of the stip 52. The material 56 may be formed to provide a relatively smooth rounded tip 31 for the positioner. The materials 55 and 56 are then allowed to cure, leaving the stip 52 fully embedded within the tip 31 of the positioner 30. The protruding portion 52a of the strip 52 should be approximately aligned with a lower edge 57 of the positioner 30. Such alignment facilitates insertion of the protruding strip 52 into the cuff 12′ when a connection between the positioner 30 and electrode 20 is desired.

The electrode/positioner assembly, i.e., the electrode 20 with cuff 12 or 12′ and positioner 30 held by the cuff 12 or 12′, may be shipped in suitable sterile packaging from the manufacturer to the operating room (OR) location where such will be inserted into a patient's cochlea or other body cavity by a surgeon.

Less common, but alternatively, the electrode 20 with cuff 12 or 12′ attached may be shipped separately from the positioner 30, and the two (electrode and positioner) may be suitably detachably joined together in the operating room (OR) by the surgeon prior to performing the electrode insertion.

Thus, from the above description, it is seen that the present invention provides an electrode/positioner system, e.g., a cochlear electrode/positioner system, that includes: (1) an electrode, e.g., a cochlear electrode, having a distal tip and a multiplicity of spaced-apart electrode contacts exposed along at least one side thereof; (2) a band (or cuff, or equivalent) secured near the distal tip of the electrode; and (3) a positioner having a distal tip adapted to be detachably connected to the band (or cuff). The distal tip of the positioner is connected to the band when an advancing force is applied to the positioner, and the distal tip of the positioner is detached from the band when a retracting force is applied to the positioner. As used herein, an “advancing force” is one wherein the positioner is advanced or moved in a forward direction (towards the distal end of the electrode or positioner), and a “retracting force” is one wherein the positioner is retracted or moved in a rearward direction (towards the proximal end of the electrode or positioner).

While the invention herein disclosed has been described by means of specific embodiments and applications thereof, numerous modifications and variations could be made thereto by those skilled in the art without departing from the scope of the invention set forth in the claims.

What is claimed is:

1. An electrode/positioner system (10) comprising:
an electrode (20) having a distal tip (21) and a plurality of spaced-apart electrode contacts (22) along its length;
a positioner (30) having a distal tip (31), wherein the positioner is adapted to help position the electrode contacts of the electrode in a desired tissue-stimulating location; and
a cuff (12) secured to the electrode (20) near the distal tip (21) of the electrode, wherein the cuff detachably engages with a distal tip of the positioner.

2. The electrode/positioner system of claim 1 wherein the cuff comprises a ring of material through which both the electrode and distal tip of the positioner pass, wherein the cuff encircles the electrode and the distal tip of the positioner.

3. The electrode/positioner system of claim 2 wherein the cuff comprises a section of thin-walled silastic tubing that is stretchable like a rubber band and that exerts a radial force which tends to hold the distal tip of the positioner securely within the cuff when stretched.

4. The electrode/positioner system of claim 3 wherein the section of silastic tubing from which the cuff is made comprises a thin-walled silastic tube having a wall thickness of between about 100 to 150 microns.

5. The electrode/positioner system of claim 3 wherein the section of silastic tubing from which the cuff is made has an un-stretched inside diameter that is approximately the same as an outside diameter of the electrode.

6. The electrode/positioner system of claim 3 wherein the distal tip of the positioner has a cross-sectional area that is tapered, whereby the cuff is stretched more and exerts a greater radial force against the positioner as the positioner is slid deeper into the cuff.

7. The electrode/positioner system of claim 1 wherein the distal tip of the positioner includes a protruding flat portion (52a) adapted to slide into the cuff.

8. The electrode/positioner system of claim 7 wherein the positioner includes a lumen (54) passing longitudinally therethrough, and wherein the protruding flat portion is integral with a body portion (52b) inserted into a distal end of the lumen, and wherein the distal end of the lumen is closed with a fluid adhesive material which, when cured, bonds to the material from which the positioner is made, whereby the body portion becomes embedded within the distal tip of the positioner.

9. The electrode/positioner system of claim 8 wherein the body portion of the metal strip further includes a bent portion (52c) that anchors the protruding flat portion within the distal tip of the positioner.

10. An electrode/positioner system adapted for insertion into a human cochlea, comprising:
a cochlear electrode having a distal tip and a multiplicity of spaced-apart electrode contacts exposed along at least one side thereof;
a band secured near the distal tip of the cochlear electrode;
a positioner having a distal tip adapted to be detachably connected to the stretchable band, wherein the distal tip of the positioner is connected to the band when an advancing force is applied to the positioner, and wherein the distal tip of the positioner is detached from the band when a retracting force is applied to the positioner, wherein an advancing force is one wherein the positioner is advanced in a distal direction, and wherein a retracting force is one wherein the positioner is retracted in a proximal direction.

11. The electrode/positioner system of claim 10 wherein the distal tip of the positioner includes a metal strip protruding therefrom, wherein said metal strip is inserted under said band when said positioner is connected to the cochlear electrode, and wherein said metal strip is pulled out from said band when said positioner is detached from said cochlear electrode.

12. The electrode/positioner system of claim 11 wherein the metal stip comprises a strip made substantially from platinum.

13. The electrode/positioner system of claim 10 wherein the band comprises a stretchable band.

14. The electrode/positioner system of claim 13 wherein the stretchable band is secured to the cochlear electrode at a location that is between about 3 and 6 mm from the distal tip of the electrode.

15. The electrode/positioner system of claim 13 wherein the stretchable band is made from a section of thin-walled silastic tube, the silastic tube having a wall thickness of between about 100 and 150 microns.

16. An electrode/positioner system adapted for insertion into body tissue, comprising:
an electrode having a distal tip and a multiplicity of spaced-apart electrode contacts exposed along at least one side of the electrode;
a loop secured to the electrode near the distal tip of the electrode;
a positioner having a distal tip, wherein the distal tip of the positioner is adapted to be engaged with the loop when an advancing force is applied to the positioner, and wherein the distal tip of the positioner is adapted to be disengaged from the loop when a retracting force is applied to the positioner.

17. A tool (40) for placing a cuff (12) near a distal end of a cochlear electrode, the cuff comprising a stretchable band having an inside surface and being made from a biocompatible material, the tool comprising reverse action tweezers having a handle portion (45) and distal fingers (42, 43) adapted to hold the cuff on the inside surface thereof, wherein the distal fingers separate from each other as opposing sides of the handle portion (45) are squeezed together, thereby stretching the cuff, and wherein at least one of the distal fingers (43) includes a slot (46) therein, said slot facilitating access to the inside surface of the cuff so as to allow a suitable bonding agent to be applied thereto.

18. The tool of claim 17 further including an adjustable knob (44) that sets a minimum separation distance of the fingers (42, 43) when no squeezing force is applied to the handle of the tool.
